(19) United States
(12) Patent Application Publication
(10) Pub. No.: US 2011/0257661 A1
(43) Pub. Date: Oct. 20, 2011

(54) SURGICAL ROBOT FOR LIPOSUCTION

(76) Inventors: Seung Wook Choi, Gyeonggi-do (KR); Min Kyu Lee, Gyeonggi-do (KR); Min Hwa Lee, Seoul (KR)

(21) Appl. No.: 13/140,988

(22) PCT Filed: Jan. 19, 2010
(86) PCT No.: PCT/KR2010/000340

§ 371 Date: Jun. 20, 2011

(30) Foreign Application Priority Data
Jan. 20, 2009 (KR) 10-2009-0004788
Jan. 20, 2009 (KR) 10-2009-0004789
Feb. 10, 2009 (KR) 10-2009-0010455

(51) Int. Cl. A61B 19/00

(52) U.S. Cl. 606/130

(57) ABSTRACT

A liposuction surgical robot is disclosed. The liposuction surgical robot, which includes: a control unit, a robot arm driven by a particular control signal received from the control unit, a cannula mounted on the robot arm and extending in one direction, and a suction unit, which is formed on an end portion of the cannula and which is inserted into a surgical site to suction fat, allows the surgeon to perform liposuction surgery conveniently without exerting too much strength. Also, the liposuction operation can be performed with greater convenience, accuracy, and safety, by automatically controlling the surgical robot using a haptic sensor, angle sensor, etc., analyzing visual information obtained from a vision unit to automatically set surgery range, and by mounting an image-taking camera on a front end of the cannula and automatically controlling the operation of the suction unit by analyzing the obtained visual information.

### CROSS-REFERENCE TO RELATED APPLICATIONS

This application is the National Phase of PCT/KR2010/000340 filed on Jan. 19, 2010, which claims priority under 35 U.S.C. 119(a) to Patent Application No. 10-2009-0004788 filed in the Republic of Korea on Jan. 20, 2009, Patent Application No. 10-2009-0004789 filed in the Republic of Korea on Jan. 20, 2009, and Patent Application No. 10-2009-0010455 filed in the Republic of Korea on Feb. 10, 2009, all of which are hereby expressly incorporated by reference into the present application.

### BACKGROUND

The present invention relates to a surgical robot for liposuction and to a liposuction surgical robot system.

With improvements in the standard of living, the attention drawn by liposuction surgery for treating abdominal obesity, etc., is rapidly growing. Liposuction refers to a surgery operation in which a cannula, shaped as a long tube, is inserted into subcutaneous fat, and a negative pressure of 0.5 to 1.0 atm is applied, to suction fat cells that have been enlarged within the body. That is, where a person's skin is made up of the outermost skin layer, an intermediate fat layer, and an inner muscle layer, liposuction surgery suctions and discharges from the fat layer between the skin layer and muscle layer.

This liposuction surgery can remove fat tissues from not only the abdomen, thighs, hips, calves, ankles, and arms, but also from the face and neck, and is widely used even in removing gynecomastia or lipomas, etc.

A conventional liposuction operation involves making a small hole in the skin where fat is to be suctioned and inserting a cannula 12, and afterwards suctioning the fat by moving the cannula 10 as indicated by the arrows. The cannula 12 is shaped as a tubing and extracts fat by suction, supplying a negative pressure inside the cannula 12.

A liposuction operation typically takes about two or three hours, and throughout the surgical procedure, the surgeon, while holding the cannula body 14 with his/her hand, has to move the cannula to forward, backward, left, right, up, and down, keeping the cannula tip at the portion where fat is to be suctioned. Thus, from the viewpoint of the surgeon performing the operation, it is a very strenuous operation. Although an electrically-powered cannula has recently become available to reduce the arm movement of the surgeon, this has on the other hand increased the weight on the cannula body and hence does not improve the convenience of the operation.

The information in the background art described above was obtained by the inventors for the purpose of developing the present invention or was obtained during the process of developing the present invention. As such, it is to be appreciated that this information did not necessarily belong to the public domain before the patent filing date of the present invention.

### SUMMARY

An aspect of the invention is to provide a surgical robot with which a surgeon can perform liposuction surgery conveniently without exerting too much strength.

One aspect of the invention provides a surgical robot for liposuction that includes: a control unit; a robot arm driven by a particular control signal received from the control unit; a cannula mounted on the robot arm and extending in one direction; and a suction unit, which is formed on an end portion of the cannula and which is inserted into a surgical site to suction fat.

The cannula can be inserted up to a particular point on the cannula through a hole perforated in a skin of a surgery patient, and the robot arm can be driven such that the particular point on the cannula is a RCM (remote center of motion). In this case, an angle sensor can further be included to measure an angle by which the cannula is inserted, and the control unit can receive a signal from the angle sensor to generate a control signal for driving the robot arm such that the cannula remains in a particular angle range.

The surgical robot can further include a haptic sensor that measures a reaction force applied on a front end of the cannula, and the control unit can receive a signal from the haptic sensor to generate a control signal for limiting a drive range of the robot arm. In this case, the control unit can be connected to a user manipulation unit, and the control unit can receive a signal from the haptic sensor to generate a control signal for causing the user manipulation unit to supply an alarm signal. Also, the control unit can perform an override function for prioritizing a signal received by user manipulation over a signal received from the haptic sensor. Also, the control unit can perform a force feedback function for applying a reaction force to a manipulation of the user manipulation unit in correspondence to a signal received from the haptic sensor. The control unit can also generate a control signal for driving the robot arm such that the suction unit moves within an operating scope set based on a signal received from the haptic sensor.

A front end of the cannula can be processed as a gently curved surface so as not to damage the surgical site.

A vision unit can further be included, which photographs the surgical site to provide visual information, and the control unit can generate a control signal for driving the robot arm such that the suction unit moves within an operating scope set based on the visual information. In this case, the operating scope can be set from data deduced by an image analysis of the visual information. The control unit can also generate a control signal for driving the robot arm such that the suction unit moves within an operating scope set beforehand by a user.

An image-taking camera can be mounted on a front end of the cannula, and the suction unit can be operated in correspondence to visual information received from the image-taking camera. In this case, a water jet unit that shoots water to the surgical site can be coupled to an end portion of the cannula, and the water jet unit can be operated in correspondence to the visual information. Also, a computing unit can be included which determines the degree of blood loss from data deduced by an image analysis of the visual information, and the suction unit can be operated in correspondence to the determination result of the computing unit.

The computing unit can determine the degree of blood loss from the data deduced by an image analysis of the visual information obtained from an image-taking camera, which photographs the substances suctioned by the suction unit and discharged out of the body, or from data obtained by a chemical sensor, which detects a chemical component of the substances discharged from the body.

A breaker unit can be formed on an end portion of the cannula that breaks up the fat suctioned through the suction unit, and the breaker unit can be formed such that it is rotated by a suction force applied in the cannula. The cannula can be structured to bend at a particular point on the cannula.

The cannula can be vibrated at a particular frequency so as to improve the suction efficiency of the suction unit. The surgical robot can further include: a storage unit that stores data related to an amount of fat at the surgical site; a volume sensor that measures the amount of fat which is suctioned by the suction unit and discharged out from the body; and a calculating unit that deduces the degree to which surgery has progressed by comparing the data stored in the storage unit with the data measured by the volume sensor.

Another aspect of the invention provides a recorded medium readable by a surgical robot that tangibly embodies a program of instructions executable by the surgical robot, for a surgical robot for performing liposuction surgery by way of a cannula mounted on a robot arm, where the cannula extends in one direction and has a suction unit formed on one end. The program includes: (a) generating a first control signal for driving the robot arm such that the suction unit is inserted into a surgical site; (b) generating a second control signal for operating the suction unit to suction fat from a surgical site; and (c) generating a third control signal for driving the robot arm such that the suction unit moves while suctioning fat within a particular operating scope.

The surgical robot can further include a vision unit that photographs the surgical site to provide visual information, and the operating scope can be set from the data deduced by an image analysis of the visual information. Also, the surgical robot can further include a haptic sensor that measures the reaction force applied on a front end of the cannula, and the operating scope can be set based on a signal received from the haptic sensor. An image-taking camera, configured to photograph the surgical site to provide visual information, can be mounted on a front end of the cannula, while the surgical robot can further include a computing unit that determines the degree of blood loss from the data deduced by an image analysis of the visual information, and the third control signal can be generated in correspondence to the determination result of the computing unit. Operation (c) of generating the third control signal can include calculating the degree to which surgery has progressed by comparing data stored beforehand regarding the amount of fat at the surgical site with data obtained by measuring the amount of fat that is suctioned by the suction unit and discharged out from the body.

Still another aspect of the invention provides a method of performing liposuction surgery by mounting a cannula on a robot arm, where the cannula extends in one direction and has a suction unit formed on one end. The method includes: (a) driving the robot arm such that the suction unit is inserted into a surgical site; (b) operating the suction unit to suction fat from a surgical site; and (c) driving the robot arm such that the suction unit moves while suctioning fat within a particular operating scope.

The operating scope can be set from data deduced by an image analysis of visual information obtained by photographing the surgical site, or from data deduced by measuring a reaction force applied on a front end of the cannula, while operation (c) of driving the robot arm can be performed in correspondence to the determination result obtained after determining the degree of blood loss from data deduced by an image analysis of visual information obtained by an image-taking camera mounted on a front end of the cannula. Operation (c) of driving the robot arm can include calculating the degree to which surgery has progressed by comparing data stored beforehand regarding the amount of fat at the surgical site with data obtained by measuring the amount of fat that is suctioned by the suction unit and discharged out from the body.

Yet another aspect of the invention provides a liposuction surgical robot system that includes: a control unit; a robot arm driven by a particular control signal received from the control unit; a magnet unit mounted on the robot arm; and a cannula module, which includes a magnetic object to correspond with a magnetic force of the magnet unit, and which is inserted into the surgical site to suction fat.

The control unit can generate a control signal for moving the magnet unit near to a surgical site, so that the cannula module is moved by magnetic force in correspondence to a movement of the magnet unit, and the cannula module can include a permanent magnet.

A tube can be connected to the cannula module that serves as a passageway through which suctioned fat is discharged out from the body. The tube can be made of a flexible material and can be detachably coupled to the cannula module.

A haptic sensor can further be included which measures a reaction force applied on a front end of the cannula module, while the control unit can receive a signal from the haptic sensor to generate a control signal for limiting a drive range of the robot arm. In this case, the control unit can be connected to a user manipulation unit, and the control unit can receive a signal from the haptic sensor to generate a control signal for causing the user manipulation unit to supply an alarm signal. Also, the control unit can perform an override function for prioritizing a signal received by user manipulation over a signal received from the haptic sensor. Also, the control unit can perform a force feedback function for applying a reaction force to a manipulation of the user manipulation unit in correspondence to a signal received from the haptic sensor. Furthermore, the control unit can generate a control signal for driving the robot arm such that the cannula module moves within an operating scope set based on a signal received from the haptic sensor.

A front end of the cannula module can be processed as a gently curved surface so as not to damage a surgical site.

The liposuction surgical robot system can further include a vision unit that photographs the surgical site to provide visual information, while the control unit can generate a control signal for driving the robot arm such that the cannula module moves within an operating scope set based on the visual information. In this case, the operating scope can be set from data deduced by an image analysis of the visual information. The control unit can generate a control signal for driving the robot arm such that the cannula module moves within an operating scope set beforehand by a user.

An image-taking camera can be mounted on a front end of the cannula module, and the control unit can generate a control signal for driving the robot arm such that the cannula module moves in correspondence to visual information received from the image-taking camera. In this case, a water jet unit that shoots water to the surgical site can be coupled to the cannula module, and the water jet unit can be operated in correspondence to the visual information. Also, a computing unit can further be included that determines the degree of blood loss from data deduced by an image analysis of the visual information, and the cannula module can be operated in correspondence to the determination result of the computing unit.

The computing unit can determine the degree of blood loss from the data deduced by an image analysis of the visual information obtained from an image-taking camera, which photographs the substances suctioned by the suction unit and discharged out of the body, or from data obtained by a chemical sensor, which detects a chemical component of the substances discharged from the body.

The cannula module can include a breaker unit that breaks up the suctioned fat, and the breaker unit can be formed such that it is rotated by a suction force applied in the cannula module.

Another aspect of the invention provides a cannula module that includes: a housing, which is formed in a tubing shape and which includes a magnetic object such that the housing can move in correspondence to an external magnetic force; a suction inlet perforated in a surface of the housing to suction fat at a surgical site; and a suction outlet, which is perforated in a surface of the housing, communicates with the suction inlet, and is connected with a suction line for forming a negative pressure within the housing.

A front end of the housing can be processed as a gently curved surface so as not to damage the surgical site, and a breaker unit for breaking up the fat suctioned through the suction inlet can be coupled inside the housing.

The cannula module can vibrate at a particular frequency so as to improve suction efficiency.

A storage unit for storing data related to an amount of fat at a surgical site, a volume sensor for measuring the amount of fat suctioned by the cannula module and discharged out from the body, and a calculating unit for deducing the degree to which surgery has progressed by comparing data stored in the storage unit with data measured by the volume sensor, can further be included.

Still another aspect of the invention provides a recorded medium readable by a surgical robot system that tangibly embodies a program of instructions executable by the surgical robot system, for a surgical robot system for performing liposuction surgery by inserting a cannula module, which includes a magnetic object, into a surgical site and driving a robot arm, on which a magnet unit is mounted. The program includes: (a) generating a first control signal for driving the robot arm such that the cannula module is moved by a magnetic force of the magnet unit; (b) generating a second control signal for operating the cannula module to suction fat from a surgical site; and (c) generating a third control signal for driving the robot arm such that the cannula module moves while suctioning fat within a particular operating scope.

The surgical robot system can further include a vision unit that photographs the surgical site to provide visual information, and the operating scope can be set from data deduced by an image analysis of the visual information. Also, the surgical robot system can further include a haptic sensor that measures a reaction force applied on a front end of the cannula module, and the operating scope can be set based on a signal received from the haptic sensor. Furthermore, an image-taking camera that photographs the surgical site to provide visual information can be mounted on a front end of the cannula module, and the surgical robot system can further include a computing unit that determines the degree of blood loss from data deduced by an image analysis of the visual information, while the third control signal can be generated in correspondence to the determination result of the computing unit. Operation (c) of generating the third control signal can include calculating the degree to which surgery has progressed by comparing data stored beforehand regarding the amount of fat at the surgical site with data obtained by measuring the amount of fat suctioned by the cannula module and discharged out from the body.

Yet another aspect of the invention provides a method of performing liposuction surgery by driving a robot arm on which a magnet unit is mounted. The method includes: (a) inserting a cannula module, which includes a magnetic object, into a surgical site; (b) operating the cannula module to suction fat from a surgical site; and (c) driving the robot arm to move the magnet unit near a surgical site such that the cannula module moves in correspondence to a movement of the magnet unit while suctioning fat within a particular operating scope.

The operating scope can be set from data deduced by an image analysis of visual information obtained by photographing the surgical site, or from data deduced by measuring a reaction force applied on a front end of the cannula module, and operation (c) of driving the robot arm can be performed in correspondence to the determination result obtained after determining the degree of blood loss from data deduced by an image analysis of the visual information obtained by an image-taking camera mounted on a front end of the cannula module. Operation (c) of driving the robot arm can include calculating the degree to which surgery has progressed by comparing data stored beforehand regarding the amount of fat at a surgical site with data obtained by measuring the amount of fat suctioned by the cannula module and discharged out from the body.

Another aspect of the invention provides a surgical robot for liposuction that includes: a control unit; a robot arm driven by a particular control signal received from the control unit; a cannula mounted on the robot arm and extending in one direction; a suction unit that is formed on an end portion of the cannula and is inserted into a surgical site to suction fat; and an ultrasound transducer coupled to a front end of the cannula.

The cannula can be inserted up to a particular point on the cannula through a hole perforated in a skin of the surgery patient, and the robot arm can be driven such that the particular point on the cannula is a RCM (remote center of motion).

An angle sensor can further be included which measures an angle by which the cannula is inserted, and the control unit can receive a signal from the angle sensor to generate a control signal for driving the robot arm such that the cannula remains in a particular angle range. Or, the control unit can obtain information related to an angle by which the cannula is inserted, from information related to a drive status of the robot arm, to generate a control signal for driving the robot arm such that the cannula remains in a particular angle range.

A front end of the cannula can be processed as a gently curved surface so as not to damage a surgical site.

The control unit can generate a control signal for driving the robot arm such that the suction unit moves within an operating scope set beforehand by a user, and can perform an override function for prioritizing a signal received by user manipulation over a signal received from the ultrasound transducer.

The ultrasound transducer can provide visual information by photographing the surgical site, and the control unit can generate a control signal for driving the robot arm such that the suction unit moves within an operating scope set based on the visual information. In this case, the operating scope can be set from data deduced by an image analysis of the visual information.

Also, the ultrasound transducer can provide a Doppler signal in correspondence to the surgical site, and the control unit can generate a control signal for driving the robot arm such that the suction unit moves within an operating scope set based on the Doppler signal.

In this case, the control unit can be connected to a user manipulation unit, and the control unit can generate a control signal for causing the user manipulation unit to supply an alarm signal if the suction unit moves outside the operating scope.

The ultrasound transducer can be of a point sensor type or a line sensor type, and the control unit can generate a control signal for driving the robot arm in such a way that enables the ultrasound transducer to obtain forward visual information while moving. The ultrasound transducer can emit ultrasonic waves that provide enough energy to heat and liquefy the fat at the surgical site.

Yet another aspect of the invention provides a surgical robot for liposuction that includes: a control unit; a robot arm driven by a particular control signal received from the control unit; a cannula mounted on the robot arm that extends in one direction; a suction unit, which is formed on an end portion of the cannula, and which is inserted into a surgical site to suction fat; and a reaction sensing unit that measures a reaction force applied on a front end of the cannula.

The control unit can receive a signal from the reaction sensing unit to generate a control signal for limiting a drive range of the robot arm. The reaction sensing unit can be positioned between the robot arm and the cannula or at the middle of the cannula, or can be built in inside the front end of the cannula.

Still another aspect of the invention provides a recorded medium readable by a surgical robot that tangibly embodies a program of instructions executable by the surgical robot, for a surgical robot having a cannula, which extends in one direction, has a suction unit formed on one end for suctioning fat, and has an ultrasound transducer for photographing a surgical site and providing visual information coupled to its front end, mounted on a robot arm for performing liposuction surgery. The program includes: (a) generating a first control signal for driving the robot arm such that the suction unit is inserted into a surgical site; (b) generating a second control signal for operating the suction unit to suction fat from a surgical site; and (c) generating a third control signal for driving the robot arm such that the suction unit moves within a particular operating scope, which is set from data deduced by an image analysis of the visual information.

Yet another aspect of the invention provides a method of performing liposuction surgery by mounting on a robot arm a cannula, which extends in one direction, has a suction unit formed on one end for suctioning fat, and has an ultrasound transducer coupled to its front end for photographing a surgical site and providing visual information. The method includes: (a) driving the robot arm such that the suction unit is inserted into a surgical site; (b) operating the suction unit to suction fat from a surgical site; and (c) driving the robot arm such that the suction unit moves within a particular operating scope, which is set from data deduced by an image analysis of the visual information.

A preferred embodiment of the invention can enable a surgeon to perform liposuction surgery conveniently without exerting too much strength, by mounting a liposuction cannula onto a robot arm and driving the robot arm or by manufacturing the liposuction cannula as a module made with a magnetic object, inserting the module into the body, and driving a robot arm equipped with a magnet to move the cannula module.

Also, a liposuction operation can be performed with greater convenience, accuracy, and safety, by automatically controlling the surgical robot using a haptic sensor, angle sensor, etc., analyzing visual information obtained from a vision unit to automatically set surgery range, and by mounting an image-taking camera on a front end of the cannula and automatically controlling the operation of the suction unit by analyzing the obtained visual information.

Furthermore, the liposuction operation can also be performed with greater convenience, accuracy, and safety, by mounting an ultrasound transducer on a front end of the cannula, so that the surgical robot may be automatically controlled based on the visual information received therefrom, the surgery range may be set automatically, and the fat may be suctioned after being liquefied by ultrasonic waves.

Additional aspects, features, and advantages, other than those described above, will be apparent from the claims and written description below.

### BRIEF DESCRIPTION OF THE DRAWINGS

FIG. 1 illustrates a liposuction surgery procedure according to the related art.

FIG. 2 is a schematic view of a surgical robot for liposuction according to an embodiment of the invention.

FIG. 3 is a schematic view of a surgical robot for liposuction according to an embodiment of the invention.

FIG. 4 is a magnified view of an end portion of a cannula according to an embodiment of the invention.

FIG. 5 is a magnified view of an end portion of a cannula according to an embodiment of the invention.

FIG. 6 is a flowchart illustrating a method of performing liposuction surgery according to an embodiment of the invention.

FIG. 7 is a schematic view of a liposuction surgical robot system according to an embodiment of the invention.

FIG. 8 illustrates a cannula module according to an embodiment of the invention.

FIG. 9 is a flowchart illustrating a method of performing liposuction surgery according to an embodiment of the invention.

FIG. 10 is a schematic view of a surgical robot for liposuction according to an embodiment of the invention.

FIG. 11 is a flowchart illustrating a method of performing liposuction surgery according to an embodiment of the invention.

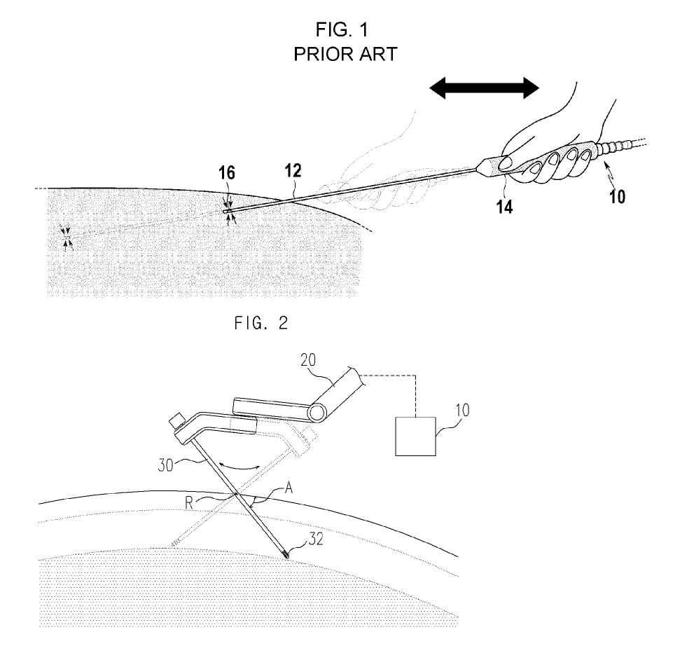

### DETAILED DESCRIPTION

As the present invention allows for various changes and numerous embodiments, particular embodiments will be illustrated in the drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention. In the written description, certain detailed explanations of related art are omitted when it is deemed that they may unnecessarily obscure the essence of the present invention.

While such terms as “first” and “second,” etc., may be used to describe various components, such components must not be limited to the above terms. The above terms are used only to distinguish one component from another.

The terms used in the present specification are merely used to describe particular embodiments, and are not intended to limit the present invention. An expression used in the singular encompasses the expression of the plural, unless it has a clearly different meaning in the context. In the present specification, it is to be understood that the terms “including” or “having,” etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Certain embodiments of the present invention will be described below in detail with reference to the accompanying drawings. Those components that are the same or are in correspondence are rendered the same reference numeral regardless of the figure number, and redundant descriptions are omitted.

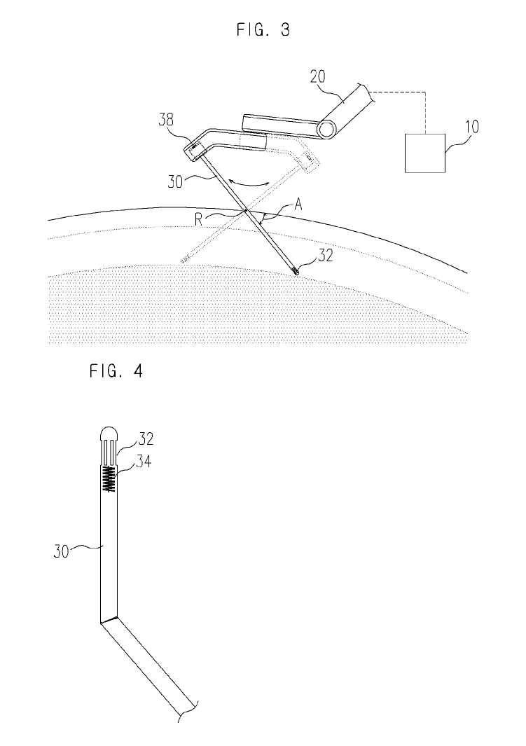

FIG. 2 is a schematic view of a surgical robot for liposuction according to an embodiment of the invention, and FIG. 3 is a schematic view of a surgical robot for liposuction according to an embodiment of the invention. Illustrated in FIG. 2 and FIG. 3 are a control unit 10, a robot arm 20, a cannula 30, a suction unit 32, and a haptic sensor 38.

In this embodiment, the cannula for liposuction may be mounted on a surgical robot arm, instead of being held by a person, so that various automatic control functions of the surgical robot may be utilized to perform the liposuction surgery with greater convenience, accuracy, and safety.

That is, the structure of a robot according to this embodiment may include a cannula 30 mounted on a robot arm 20, which may be driven by receiving a control signal from a control unit 10, where the cannula 30 may extend in one direction and on its end portion have a suction unit 32 formed, which may be inserted into the surgical site to suction fat. The cannula 30 may be a member shaped as a tubing, and by using a tubing that has a diameter of 4 mm or smaller, it is possible not to leave a scar during the insertion through the skin.

The control unit 10 according to this embodiment may be the part that generates and transmits control signals for driving the surgical robot and can be implemented in the form of a microprocessor-equipped robot or computer, etc. As described later on, the control unit 10 can serve to generate the control signals necessary for driving the robot by receiving signals transmitted from the manipulation of the user or from various sensors.

It can also be implemented with an integrated processor, which may receive, process, and analyze visual information obtained from a vision unit or an image-taking camera, and control the suction operation, etc., applied in the cannula 30.

When performing surgery by mounting a cannula 30 on a surgical robot arm 20, problems may arise where the movement of the cannula 30 damages the patient's skin, or conversely, the patient's skin inhibits the free movement of the cannula 30.

Thus, a particular position on the cannula 30 can be set as an imaginary center point of rotation and movement, and the robot arm 20 can be controlled such that the cannula 30 moves and rotates about this point, where this imaginary center point is referred to as a “remote center of motion (RCM).”

That is, the cannula 30 mounted on the robot arm 20 according to this embodiment may be inserted, during the surgical procedure, through a hole perforated in the patient's skin up to a particular point (“R” of FIG. 2) on the cannula 30, and by driving the robot arm 20 with an RCM function such that this point becomes the remote center of motion, the cannula 30 mounted on the robot arm 20 can be moved and rotated freely to perform the liposuction operation. In this case, the cannula 30 mounted on the robot arm 20 may suction fat while freely moving forward, backward, left, right, up, and down, according to the driving of the robot arm 20. The cannula 30 can suction fat while moving and rotating about the RCM point, centering on the hole perforated in the patient's skin, for example while rotating 360 degrees to draw a cone-like path.

Of course, it is not absolutely necessary that a robot arm with which an RCM is implemented according to this embodiment have the shape and structure illustrated in FIG. 2 and FIG. 3.

If the angle by which the cannula 30 is inserted through the skin is equal to or greater than a particular angle (e.g. “A” of FIG. 2), an accident can occur in which the front end of the cannula 30 invades a muscle. Thus, by installing an angle sensor (not shown) capable of detecting the angle by which the cannula 30 is inserted through the skin, and receiving signals from the angle sensor, the robot arm 20 can be driven such that the cannula 30 does not move beyond a particular angle, i.e. the cannula 30 moves only within a preset angle range.

If the cannula 30 is moved excessively during a liposuction procedure, there is a risk of damage incurred on the skin layer or muscle layer, other than the fat layer. To prevent this, a front end of the cannula 30, i.e. the tip portion, can be manufactured in a blunt shape or processed into a gently curved surface, so that the surgical site is not excessively damaged, with no penetration into the skin layer or muscle layer, etc., even when an inordinate amount of force is applied, up to a certain degree.

Also, to prevent this, a haptic sensor 38 can be installed in a suitable position, such as at the portion where the cannula 30 is mounted on the robot arm 20, to sense the reaction force applied on the front end of the cannula 30, as illustrated in FIG. 3. The control unit 10 controlling the robot arm 20 can receive the sensing signals from the haptic sensor 38, and if a force greater than a predefined amount is applied on the tip portion of the cannula 30, such as when the tip portion of the cannula 30 touches a skin tissue or a muscle tissue, can limit the drive range of the robot arm 20 so as not to further advance the cannula 30.

This uses the property that the reaction force applied on the cannula 30 differs according to the portion touched by the tip of the cannula 30, because the skin and muscle are considerably tough whereas fat tissues are relatively soft. As the haptic sensor 38 detects the reaction force on the tip of the cannula 30, the surgical robot is able to automatically sense skin and muscle, whereby the liposuction operation can be performed with the robot arm 20 automatically moving the cannula 30.

A user manipulation unit can be connected to a surgical robot according to this embodiment, in which case the surgeon can control the robot arm 20 to move forward, backward, left, right, up, and down using a 3-dimensional joystick, etc., installed on the user manipulation unit. If the front end portion of the cannula 30 touches skin or muscle instead of fat when the surgeon is manipulating the robot, the haptic sensor 38 may sense this, and a suitable alarm signal, such as a warning sound, etc., may be supplied at the user manipulation unit in accordance to a signal from the haptic sensor 38, so as not to further advance the cannula 30.

By thus sensing skin, fat, and muscle automatically, the suction unit 32 can be controlled to move only in the fat layer. Occasionally, however, there may be instances of error in which, despite the surgeon's judgment that the suction unit 32 is positioned among fat tissues, the haptic sensor 38 identifies them as skin or muscle and prevents the robot arm 20 from advancing further.

To deal with this, a surgical robot according to this embodiment can also include an “override” function, which prioritizes signals received from user manipulation over signals received from the haptic sensor 38, i.e. enables the surgeon to forcibly operate the robot when necessary.

Furthermore, a “force feedback” function can also be included, which applies a reaction force to a manipulation on the joystick, etc., installed on the user manipulation unit in accordance to a signal received from the haptic sensor 38, to recreate the sensation felt when the surgeon uses his/her own hands to operate. That is, a function can be installed by which the haptic sensor 38 senses the touching of skin or muscle instead of fat by the front end portion of the cannula 30 and reflects the force onto the joystick, etc., and as the reaction force felt at the tip of the cannula 30 can be felt at the joystick also, the sensation felt when the surgeon operates with his/her own hands can be recreated.

A surgical robot according to this embodiment can be driven by a surgeon manually manipulating a 3-dimensional joystick, etc., or can be controlled such that the suction unit 32 moves automatically to find a fat layer within a defined operating scope, or the operating scope can be pre-designated beforehand, so that the suction unit 32 only moves within this range.

Here, various control methods can be applied in designating the range of movement of the suction unit 32, i.e. the operating scope. For example, a vision unit, such as a camera or laparoscope, etc., which photographs the surgical site to provide visual information can be installed, and the operating scope can be set based on the visual information obtained from the vision unit, so that the robot arm 20 can be driven to move the suction unit 32 within that range. That is, the operating scope can be designated by marking on the screen with an input means such as a mouse or touchscreen, etc., while viewing the images photographed from the vision unit on a monitor screen.

The operating scope can also be designated by image-processing the visual information obtained from the vision unit to automatically detect the scope in which the fat layer is distributed.

Furthermore, the surgeon can input a particular range by driving the robot arm 20 beforehand, before inserting the cannula 30 into the body, and arrange the settings such that the robot arm 20 is driven only within the inputted range, so that the suction unit 32 is controlled to move only within the operating scope.

That is, before the operation, the cannula 30 can be mounted on the robot and moved manually, to input into the robot beforehand the range in which the cannula 30 can move, and during the operation, the robot arm 20 can be made to move only within the pre-inputted range. For example, a pen can be tied to the tip of the cannula 30, and a movement corresponding to drawing the range where liposuction is to be performed with the pen can be inputted, at which the robot may identify the movement, and the cannula 30 can be operated within the inputted range only.

The operating scope within which the suction unit 32 may move can also be set based on feedback signals received from the haptic sensor 38 described above.

During a liposuction surgery procedure, the surgeon can hold a joystick, etc., with the right hand to manipulate the robot, and touch the skin at the point where the tip portion of the cannula 30 is positioned with the left hand to feel whether there is skin, muscle, or fat in the current position, i.e. to check whether or not the robot is performing surgery at the correct position.

To control this automatically, a cannula 30 according to this embodiment can be mounted with a subminiature camera, etc., to check whether or not the tip of the cannula 30 is positioned among fat tissues. That is, visual information can be received from an image-taking camera mounted on the front end of the cannula 30, and the operation of the suction unit 32 can be controlled correspondingly.

When performing liposuction surgery by mounting a water jet unit on the tip portion of the cannula 30 and shooting water, for example, it is possible to control the setup to distinguish whether or not the point at which the front end of the cannula 30 is currently positioned is among fat, using the visual information obtained from the image-taking camera, and automatically shoot the water when necessary.

It is also possible to determine whether or not there is blood loss or determine the degree of blood loss, by image-processing the visual information obtained from the image-taking camera. For example, it can be determined that there is blood loss if, according to an analysis of the visual information screen, the proportion of the color red is equal to or greater than a certain degree, and the setup can be controlled to halt the movement of the robot (and/or stop the operation of the suction unit 32).

Methods of sensing blood loss for halting the movement of the robot can include, in addition to the method described above of sensing blood loss by mounting a camera on a tip of the cannula 30 and analyzing the visuals obtained therefrom, a method of installing a camera at the outlet, etc., through which the suctioned fat is discharged and sensing blood loss from the color of the discharged substances, a method of mounting a chemical sensor on the discharge outlet, etc., and sensing whether or not the proportion of blood in the suctioned fat tissues is equal to or above a particular value, and the like.

That is, determining whether or not there is blood loss or determining the degree of blood loss can be performed from the data deduced by an image analysis of the visual information obtained from an image-taking camera that photographs the substances suctioned by the suction unit and discharged out from the body, or from the data obtained by a chemical sensor that detects the chemical components of the substances discharged out of the body.

A computing unit for determining the degree of blood loss by analyzing various data can be prepared as a separate device or can also be integrated into the microprocessor for controlling the robot.

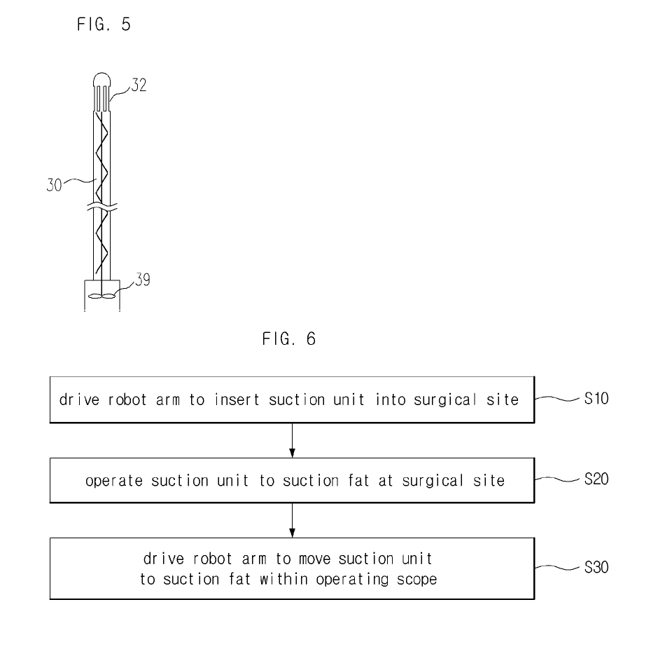

FIG. 4 is a magnified view of an end portion of a cannula according to an embodiment of the invention, and FIG. 5 is a magnified view of an end portion of a cannula according to an embodiment of the invention. Illustrated in FIG. 4 and FIG. 5 are a cannula 30, a suction unit 32, a breaker unit 34, and a propeller 39.

A suction unit 32 formed on a tip of a cannula 30 according to this embodiment can be manufactured to simply perform a suctioning function, or a breaker unit 34, such as with drill blades, etc., for breaking up the suctioned fat tissues can be formed additionally.

The breaker unit 34 can be implemented in the form of drill blades installed on the inside of the suction unit 32, as illustrated in FIG. 4, where the suctioned fat can be broken up by rotating the drill blades. The breaker unit 34 can be rotated by electrical power.

By installing a breaker unit 34 of drill blades, etc., within the cannula 30 and connecting it to a propeller 39, etc., as illustrated in FIG. 5, the propeller 39 can be rotated by the pressure difference resulting from the suction, and the drill blades within the cannula 30 can also be rotated in linkage.

A cannula 30 mounted on the robot arm 20 according to this embodiment can be an existing manual cannula used for liposuction that has been modified in structure to be mounted on the robot arm 20 or can also be manufactured as an exclusive cannula that has been designed in correspondence to the shape and structure of the robot arm 20.

The cannula 30 according to this embodiment can be structured to bend at a middle point, in which case the front end of the cannula 30, inserted through a hole perforated in the skin and bent, can be rotated freely without having to implement an RCM as in the case of a linear cannula 30, to suction fat from a larger scope.

The cannula 30 can be made to vibrate forward, backward, left, right, up, and down within a certain range, to maximize liposuction efficiency. To have the cannula 30 vibrate at a particular frequency, various methods can be applied, such as a method of adding a vibrating tool to the cannula 30, a method of implementing a vibrating function in the robot arm 20 on which the cannula 30 is mounted, and the like.

By additionally installing a volume sensor for measuring the amount of fat suctioned during the process of inserting the cannula 30 into the body and suctioning fat, it is possible to show the degree to which the operation has progressed on a monitor screen, etc.

That is, data related to the amount of fat at the surgical site can be stored beforehand in a storage unit, and the amount of suctioned fat can be measured with a volume sensor, so that by comparing the stored data with the measured data in real time, it is possible to proceed with the operation while checking the degree to which the surgery has progressed. The progress of the surgery can be displayed on a monitor, etc., installed on the user manipulation unit.

Since the amount of fat at the surgical site can be found as a 3-dimensional volume from the area of the site at which surgery is to be performed and the thickness of the fat layer, the current progress can be found by comparing the amount of fat suctioned with the overall amount of fat at the surgical site. By thus using a volume sensor to measure the amount of fat being suctioned, it is also possible to find how much fat has been suctioned at which position on the path along which the robot has moved.

FIG. 6 is a flowchart illustrating a method of performing liposuction surgery according to an embodiment of the invention.

This embodiment relates to a method of performing surgery using a surgical robot for liposuction described above. First, the robot arm 20 mounted with the cannula 30 may be driven such that an end portion of the cannula 30, i.e. the suction unit 32, is inserted in the desired surgical site (S10).

Next, the fat at the surgical site may be suctioned through the suction unit 32 as a negative pressure is applied in the cannula 30 (S20). During this procedure, the robot arm 20 may be driven such that the suction unit 32 suctions fat while moving within a designated operating scope (S30), whereby the fat may be suctioned and removed at the desired site.

The range in which the suction unit 32 moves, i.e. the operating scope, can be automatically set by photographing the surgical site with a vision unit to obtain visual information and analyzing the obtained image, as described above, whereby the surgical robot according to this embodiment can be made to detect a fat layer and perform liposuction surgery automatically.

Of course, it is not absolutely necessary that the setting of the operating scope be achieved by way of an image analysis of visual information obtained from a vision unit, and it is apparent that the operating scope within which the suction unit 32 may move can also be set based on feedback signals received from a haptic sensor 38, as already described above.

Furthermore, an image-taking camera can be mounted on a front end of the cannula 30 to obtain visual information of the site where liposuction is being performed. Using the images thus obtained, it can be determined whether or not there is blood loss or how much blood loss there is, etc., and the suction unit 32 can be controlled to operate or stop in accordance to the result. In this way, a surgical robot according to this embodiment may automatically sense blood loss to proceed with or stop the operation.

By identifying the fat at the surgical site beforehand and measuring in real time the amount of fat being suctioned by the suction unit and discharged out of the body, as described above, the measured data can be compared with the pre-identified data, so that the operation can be proceeded with while calculating and displaying the degree to which the operation has progressed.

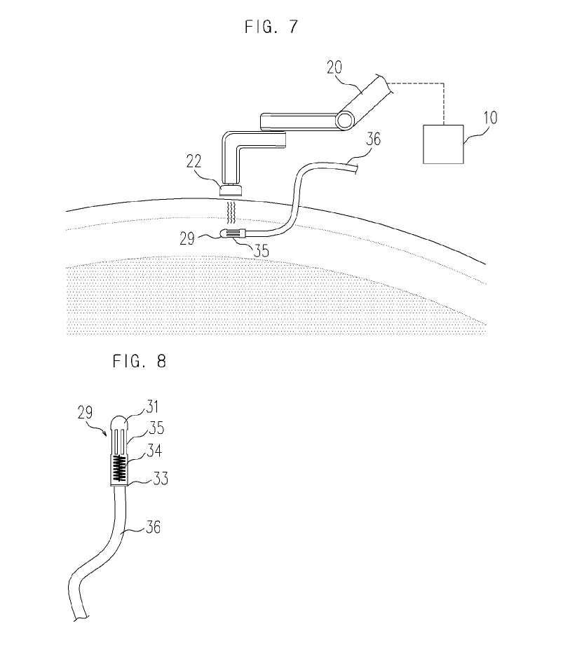

FIG. 7 is a schematic view of a liposuction surgical robot system according to an embodiment of the invention. Illustrated in FIG. 7 are a control unit 10, a robot arm 20, a magnet unit 22, a cannula module 29, a suction inlet 35, and a tube 36.

In this embodiment, instead of providing the liposuction cannula in the form of a pipe, just the suction unit part on the front end may be modularized as a separate member, which is made of a material having a magnetic quality, such as a magnet or metal, etc., and a thin, freely bendable tube 36, such as a rubber tubing, etc., may be connected to the cannula module 29.

The cannula module 29 manufactured in this manner can be inserted into the surgical site, and by moving the magnet unit 22, such as of a permanent magnet or electromagnet, etc., outside the skin, the cannula module 29 inserted into the body can be moved by way of magnetic force.

Furthermore, in this embodiment, the magnet unit 22 for moving the cannula module 29 may be mounted on the robot arm 20, so that various automatic control functions of the surgical robot may be utilized to perform the liposuction surgery with greater convenience, accuracy, and safety.

That is, the basic structure of a robot system according to this embodiment may include a robot arm 20 that may be driven by receiving a control signal from a control unit 10, a magnet unit 22 mounted on the robot arm 20, and a cannula module 29 made from a magnetic object to be able to move according to the magnetic force of the magnet unit 22.

The cannula module 29 may be a separate member that is inserted in the surgical site to suction fat, and in a portion of the cannula module 29, a suction inlet 35 for suctioning fat may be perforated. By using a tubing that has a diameter of 4 mm or smaller, it is possible not to leave a scar during the procedure of inserting the cannula module 29 through the skin.

The control unit 10 according to this embodiment may be the part that generates and transmits control signals for driving the robot arm 20 and can be implemented in the form of a microprocessor-equipped robot or computer, etc. As described later on, the control unit 10 can serve to generate the control signals necessary for driving the robot by receiving signals transmitted from the manipulation of the user or from various sensors.

It can also be implemented with an integrated processor, which may receive, process, and analyze visual information obtained from a vision unit or an image-taking camera, and control the suction operation, etc., applied on the cannula module 29.

A cannula module 29 according to this embodiment may be made of a magnetic object, and after the cannula module 29 is inserted into the body, the magnet unit 22, such as of a permanent magnet or electromagnet, etc., can be moved outside the body, whereby the cannula module 29 can be moved correspondingly by magnetic force (attractive or repulsive force).

The magnet unit 22 for thus moving the cannula module 29 can be moved manually by a person, and the magnet unit 22 can also be mounted on a robot arm 20, so that the magnet unit 22 may be moved near the surgical site by driving the robot arm 20. That is, when the part of the robot arm 20 where the magnet unit 22 is mounted is moved along the curve of the patient's skin, the cannula module 29 inserted in the body may suction fat while moving by magnetic force in accordance to the movement of the magnet unit 22.

Since a robot driven with a mounted magnet unit 22 according to this embodiment is not driven after directly invading the skin, it can omit the so-called “RCM function” of setting a remote center of motion (RCM) and having a surgical instrument rotate about this center.

A cannula module 29 according to this embodiment may be made of a magnetic object as its material, to be able to move in accordance to the magnetic force created by the magnet unit 22. Metal can be used as the magnetic object, and a permanent magnet can also be used as necessary.

If the permanent magnet used as the material for the cannula module 29 and the magnet unit 22 mounted on the robot arm 20 are arranged such that opposite poles face each other (e.g. the N-pole and S-pole), the cannula module 29 can move by attraction, and if they are arranged such that the same poles face each other (e.g. the N-pole and N-pole), the cannula module 29 can move by repulsion.

Furthermore, in cases where the magnet unit 22 mounted on the robot arm 20 is such that can alter its polarity, as with an electromagnet, etc., the electromagnet can be controlled to have the same poles or opposite poles facing each other, in relation to the permanent magnet used as material for the cannula module 29, so that the cannula module 29 may move according to attraction or repulsion as necessary.

A tube 36 can be connected to the cannula module 29 to discharge the fat suctioned through the cannula module 29, and by using a tube 36 made of a flexible material, such as with a rubber tube 36, etc., the cannula module 29 can be made to move freely within the body without being inhibited by the tube 36.

That is, a tube 36 according to this embodiment can be manufactured with a soft material such as rubber, etc., so that it may move freely within the body while connected to the cannula module 29, and may desirably be made of a material having the durability to withstand the force of pulling out the inserted cannula module 29, in order that the tube 36 may not break during the process of drawing out the cannula module 29 after surgery. Furthermore, the tube 36 according to this embodiment can also be used as a liquid-discharge tubing after surgery.

The cannula module 29 and/or tube 36 according to this embodiment can be used as an expendable product that is disposed after surgery, and by making a structure in which the tube 36 is detachably coupled to the cannula module 29, it is possible to reuse the cannula module 29 and only use the connected rubber tube 36 as an expendable product. That is, the cannula module 29 or rubber tube 36 can be manufactured for disposable or reusable usage in consideration of the material, structure, cost, etc., of each part.

If the cannula module 29 is moved excessively during a liposuction procedure, there is a risk of damage incurred on the skin layer or muscle layer, other than the fat layer. To prevent this, a front end of the cannula module 29, i.e. the tip portion, can be manufactured in a blunt shape or processed into a gently curved surface, so that the surgical site is not excessively damaged, with no penetration into the skin layer or muscle layer, etc., even when an inordinate amount of force is applied, up to a certain degree.

Also, to prevent this, a haptic sensor (not shown) can be installed in a suitable position to sense the reaction force applied on the front end of the cannula module 29. In this case, the control unit 10 controlling the robot arm 20 can receive the sensing signals from the haptic sensor, and if a force greater than a predefined amount is applied on the tip portion of the cannula module 29, such as when the tip portion of the cannula module 29 touches a skin tissue or a muscle tissue, can limit the drive range of the robot arm 20 so as not to further advance the cannula module 29.

This uses the property that the reaction force applied on the cannula module 29 differs according to the portion touched by the tip of the cannula module 29, because the skin and muscle are considerably tough whereas fat tissues are relatively soft. As the haptic sensor detects the reaction force on the tip of the cannula module 29, the surgical robot system is able to automatically sense skin and muscle, whereby the liposuction operation can be performed with the robot arm 20 automatically moving the cannula module 29.

A user manipulation unit can be connected to a surgical robot system according to this embodiment, in which case the surgeon can control the robot arm 20 to move forward, backward, left, right, up, and down using a 3-dimensional joystick, etc., installed on the user manipulation unit. If the front end portion of the cannula module 29 touches skin or muscle instead of fat when the surgeon is manipulating the robot, the haptic sensor may sense this, and a suitable alarm signal, such as a warning sound, etc., may be supplied at the user manipulation unit in accordance to a signal from the haptic sensor, to limit the driving of the robot arm 20 and prevent the cannula module 29 from advancing further.

By thus sensing skin, fat, and muscle automatically, the cannula module 29 can be controlled to move only in the fat layer. Occasionally, however, there may be instances of error in which, despite the surgeon's judgment that the cannula module 29 is positioned among fat tissues, the haptic sensor identifies them as skin or muscle and stops the robot arm 20.

To deal with this, a surgical robot system according to this embodiment can also include an “override” function, which prioritizes signals received from user manipulation over signals received from the haptic sensor, i.e. enables the surgeon to forcibly operate the robot when necessary.

Furthermore, a “force feedback” function can also be included, which applies a reaction force to a manipulation on the joystick, etc., installed on the user manipulation unit in accordance to a signal received from the haptic sensor, to recreate the sensation felt when the surgeon uses his/her own hands to operate. That is, a function can be installed by which the haptic sensor senses the touching of skin or muscle instead of fat by the front end portion of the cannula module 29 and reflects the force onto the joystick, etc., and as the reaction force felt at the tip of the cannula module 29 can be felt at the joystick also, the sensation felt when the surgeon operates with his/her own hands can be recreated.

A surgical robot system according to this embodiment can be driven by a surgeon manually manipulating a 3-dimensional joystick, etc., or can be controlled to automatically detect a fat layer and move the cannula module 29 within a defined operating scope, or the operating scope can be pre-designated beforehand, so that the cannula module 29 only moves within this range.

Here, various control methods can be applied in designating the range of movement of the cannula module 29, i.e. the operating scope. For example, a vision unit, such as a camera or laparoscope, etc., which photographs the surgical site to provide visual information can be installed, and the operating scope can be set based on the visual information obtained from the vision unit, so that the robot arm 20 can be driven to move the cannula module 29 within that range. That is, the operating scope can be designated by marking on the screen with an input means such as a mouse or touchscreen, etc., while viewing the images photographed from the vision unit on a monitor screen.

The operating scope can also be designated by image-processing the visual information obtained from the vision unit to automatically detect the scope in which the fat layer is distributed.

Furthermore, the surgeon can input a particular range by driving the robot arm 20 beforehand, before inserting the cannula module 29 into the body and moving the magnet unit 22 near the surgical site, and arrange the settings such that the robot arm 20 is driven to move the magnet unit 22 only within the inputted range, so that the cannula module 29 is controlled to move only within the operating scope.

That is, before the operation, the magnet unit 22 can be mounted on the robot arm 20 and moved manually, to input into the robot beforehand the range in which the cannula module 29 can move, and during the operation, the robot arm 20 can be made to move only within the pre-inputted range. For example, a pen can be tied to the magnet unit 22, and a movement corresponding to drawing the range where liposuction is to be performed with the pen can be inputted, at which the robot can identify the movement and move the magnet unit 22 within the inputted range only, causing the cannula module 29 to move accordingly.

The operating scope within which the cannula module 29 may move can also be set based on feedback signals received from the haptic sensor described above.

During a liposuction surgery procedure, the surgeon can hold a joystick, etc., with the right hand to manipulate the robot, and touch the skin at the point where the cannula module 29 is positioned with the left hand to feel whether there is skin, muscle, or fat in the current position, i.e. to check whether or not the cannula module 29 is performing liposuction at the correct position.

To control this automatically, a cannula module 29 according to this embodiment can be mounted with a subminiature camera, etc., to check whether or not the tip of the cannula module 29 is positioned among fat tissues. That is, visual information can be received from an image-taking camera mounted on the front end of the cannula module 29, and the movement of the cannula module 29 can be controlled correspondingly.

When performing liposuction surgery by mounting a water jet unit on the tip portion of the cannula module 29 and shooting water, for example, it is possible to control the setup to distinguish whether or not the point at which the front end of the cannula module 29 is currently positioned is among fat, using the visual information obtained from the image-taking camera, and automatically shoot the water when necessary.

It is also possible to determine whether or not there is blood loss or determine the degree of blood loss, by image-processing the visual information obtained from the image-taking camera. For example, it can be determined that there is blood loss if, according to an analysis of the visual information screen, the proportion of the color red is equal to or greater than a certain degree, and the setup can be controlled to stop the robot arm 20, so that the cannula module 29 does not move, or to stop the operation of the cannula module 29.

Methods of sensing blood loss for halting the movement of the robot can include, in addition to the method described above of sensing blood loss by mounting a camera on a tip of the cannula module 29 and analyzing the visuals obtained therefrom, a method of installing a camera at the outlet, etc., of the tube 36 through which the suctioned fat is discharged and sensing blood loss from the color of the discharged substances, a method of mounting a chemical sensor on the discharge outlet, etc., and sensing whether or not the proportion of blood in the suctioned fat tissues is equal to or above a particular value, and the like.

That is, determining whether or not there is blood loss or determining the degree of blood loss can be performed from the data deduced by an image analysis of the visual information obtained from an image-taking camera that photographs the substances suctioned by the cannula module 29 and discharged out from the body, or from the data obtained by a chemical sensor that detects the chemical components of the substances discharged out of the body.

A computing unit for determining the degree of blood loss by analyzing various data can be prepared as a separate device or can also be integrated into the microprocessor for controlling the robot arm 20.

FIG. 8 illustrates a cannula module according to an embodiment of the invention. Illustrated in FIG. 8 are a cannula module 29, a housing 31, a suction inlet 35, a suction outlet 33, a breaker unit 34, and a tube 36.

A cannula module 29 according to this embodiment may be formed in a modularized tubing shape as described above. As illustrated in FIG. 8, the cannula module 29 may include a housing 31, which forms the overall frame, one or more suction inlet 35 perforated in a surface of the housing 31, and a suction outlet 33, which communicates with the suction inlet 35, and to which a tube 36 may be coupled.

The material for the housing 31 can include a magnetic object such as metal or a permanent magnet, etc., so that the cannula module 29 can be made to move in accordance to a magnet that is moved manually or mounted on a robot arm 20, as described above. When the cannula module 29 is operated, the fat at the surgical site may be suctioned through the suction inlet 35, and the suctioned fat may be discharged out of the body through a suction line, such as a tube 36, etc., coupled to the suction outlet 33.

A cannula module 29 according to this embodiment can be manufactured to simply perform a suctioning function, or a breaker unit 34, such as with drill blades, etc., for breaking up the suctioned fat tissues can be formed additionally.

The breaker unit 34 can be implemented in the form of drill blades installed in the cannula module 29, as illustrated in FIG. 8, where the suctioned fat can be broken up by rotating the drill blades. The breaker unit 34 can be rotated by electrical power, and can also be rotated pneumatically using the suction force applied in the cannula module 29.

In cases where a breaker unit 34 is installed in the cannula module 29, just the module itself can be inserted into the body to break up fat, instead of connecting a rubber tube 36 to the cannula module 29. In such cases, the fat tissues broken up can be suctioned out of the body using a separate suction tool.

The cannula module 29 can be made to vibrate forward, backward, left, right, up, and down within a certain range, to maximize liposuction efficiency. For example, a vibrating tool can be added to the cannula module 29 to have the cannula module vibrate at a particular frequency.

By additionally installing a volume sensor for measuring the amount of fat suctioned during the process of inserting the cannula module 29 into the body and suctioning fat, it is possible to show the degree to which the operation has progressed on a monitor screen, etc.

That is, data related to the amount of fat at the surgical site can be stored beforehand in a storage unit, and the amount of suctioned fat can be measured with a volume sensor, so that by comparing the stored data with the measured data in real time, it is possible to proceed with the operation while checking the degree to which the surgery has progressed. The progress of the surgery can be displayed on a monitor, etc., installed on the user manipulation unit.

Since the amount of fat at the surgical site can be found as a 3-dimensional volume from the area of the site at which surgery is to be performed and the thickness of the fat layer, the current progress can be found by comparing the amount of fat suctioned with the overall amount of fat at the surgical site. By thus using a volume sensor to measure the amount of fat being suctioned, it is also possible to find how much fat has been suctioned at which position on the path along which the cannula module 29 has moved.

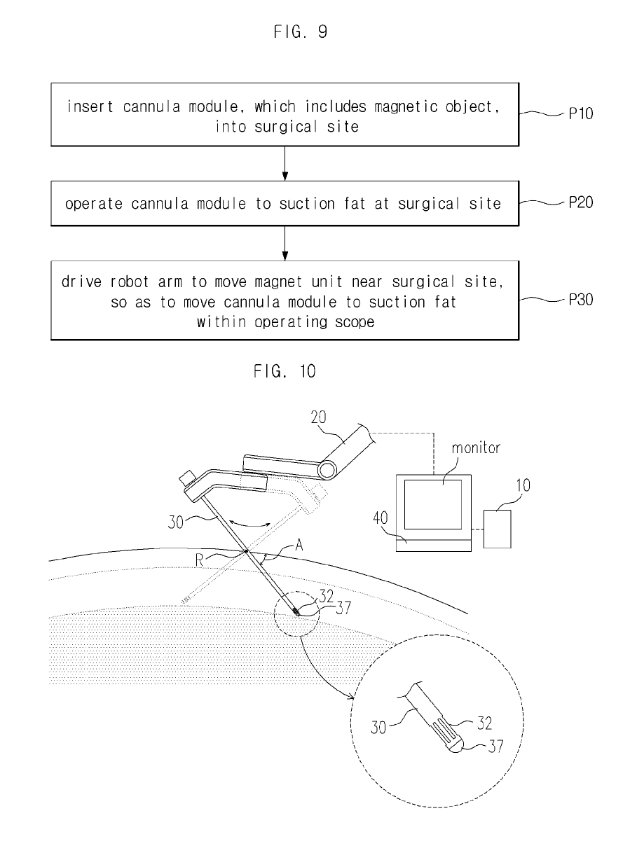

FIG. 9 is a flowchart illustrating a method of performing liposuction surgery according to an embodiment of the invention.

This embodiment relates to a method of performing surgery using a liposuction surgical robot system described above. First, the cannula module 29 made with a magnetic object may be inserted in the surgical site (P10).

Next, the fat at the surgical site may be suctioned through the suction inlet 35 as a negative pressure is applied in the cannula module 29 (P20). During this procedure, the robot arm 20 may be driven to move the magnet unit 22 near the surgical site, such that the cannula module 29 suctions fat while moving within a designated operating scope (P30), whereby the fat may be suctioned and removed at the desired site.

The range in which the cannula module 29 moves, i.e. the operating scope, can be automatically set by photographing the surgical site with a vision unit to obtain visual information and analyzing the obtained image, as described above, whereby the surgical robot system according to this embodiment can be made to detect a fat layer and perform liposuction surgery automatically.

Of course, it is not absolutely necessary that the setting of the operating scope be achieved by way of an image analysis of visual information obtained from a vision unit, and it is apparent that the operating scope within which the cannula module 29 may move can also be set based on feedback signals received from a haptic sensor, as already described above.

Furthermore, an image-taking camera can be mounted on a front end of the cannula module 29 to obtain visual information of the site where liposuction is being performed. Using the images thus obtained, it can be determined whether or not there is blood loss or how much blood loss there is, etc., and the cannula module 29 can be controlled to operate or stop in accordance to the result. In this way, a surgical robot according to this embodiment may automatically sense blood loss to proceed with or stop the operation.

By identifying the fat at the surgical site beforehand and measuring in real time the amount of fat being suctioned by the cannula module and discharged out of the body, as described above, the measured data can be compared with the pre-identified data, so that the operation can be proceeded with while calculating and displaying the degree to which the operation has progressed.

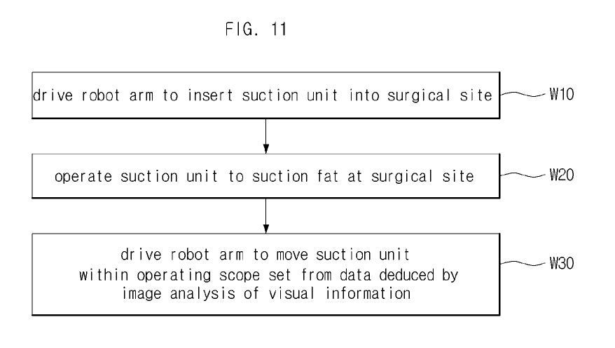

FIG. 10 is a schematic view of a surgical robot for liposuction according to an embodiment of the invention. Illustrated in FIG. 10 are a control unit 10, a robot arm 20, a cannula 30, a suction unit 32, an ultrasound transducer 37, and a user manipulation unit 40.

In this embodiment, the cannula for liposuction may be mounted on the arm of a surgical robot, instead of being held by a person, so that various automatic control functions of the surgical robot may be utilized to perform the liposuction surgery with greater convenience, accuracy, and safety.

That is, the structure of a robot according to this embodiment may include a cannula 30 mounted on a robot arm 20, which may be driven by receiving a control signal from a control unit 10, where the cannula 30 may extend in one direction and on its end portion have a suction unit 32 formed, which may be inserted into the surgical site to suction fat. The cannula 30 may be a member shaped as a tubing, and by using a tubing that has a diameter of 4 mm or smaller, it is possible not to leave a scar during the insertion through the skin.

The control unit 10 according to this embodiment may be the part that generates and transmits control signals for driving the surgical robot and can be implemented in the form of a microprocessor-equipped robot or computer, etc. As described later on, the control unit 10 can serve to generate the control signals necessary for driving the robot by receiving signals transmitted from the manipulation of the user or from various sensors.

It can also be implemented with an integrated processor, which may receive, process, and analyze visual information obtained from an ultrasound transducer 37 described later, and control the suction operation, etc., applied in the cannula 30.

When performing surgery by mounting a cannula 30 on a surgical robot arm 20, problems may arise where the movement of the cannula 30 damages the patient's skin, or conversely, the patient's skin inhibits the free movement of the cannula 30.

Thus, a particular position on the cannula 30 can be set as an imaginary center point of rotation and movement, and the robot arm 20 can be controlled such that the cannula 30 moves and rotates about this point, where this imaginary center point is referred to as a “remote center of motion (RCM).”

That is, the cannula 30 mounted on the robot arm 20 according to this embodiment may be inserted, during the surgical procedure, through a hole perforated in the patient's skin up to a particular point (“R” of FIG. 10) on the cannula 30, and by driving the robot arm 20 with an RCM function such that this point becomes the remote center of motion, the cannula 30 mounted on the robot arm 20 can be moved and rotated freely to perform the liposuction operation. In this case, the cannula 30 mounted on the robot arm 20 may suction fat while freely moving forward, backward, left, right, up, and down, according to the driving of the robot arm 20. The cannula 30 can suction fat while moving and rotating about the RCM point, centering on the hole perforated in the patient's skin, for example while rotating 360 degrees to draw a cone-like path.

Of course, it is not absolutely necessary that a robot arm with which an RCM is implemented according to this embodiment have the shape and structure illustrated in FIG. 10.

If the angle by which the cannula 30 is inserted through the skin is equal to or greater than a particular angle (e.g. “A” of FIG. 10), an accident can occur in which the front end of the cannula 30 invades a muscle. Thus, by installing an angle sensor (not shown) capable of detecting the angle by which the cannula 30 is inserted through the skin, and receiving signals from the angle sensor, the robot arm 20 can be driven such that the cannula 30 does not move beyond a particular angle, i.e. the cannula 30 moves only within a preset angle range.

Since the robot arm 20 according to this embodiment may be driven by receiving control signals from the control unit 10, it is not absolutely necessary that an angle sensor be installed to drive the robot arm 20 within a preset angle range, and it is apparent that the angle by which the cannula 30 is inserted can be reversely estimated from information regarding the drive state of the robot arm 20, to generate and transmit the control signals for operating the cannula 30 within the set angle range.

If the cannula 30 is moved excessively during a liposuction procedure, there is a risk of damage incurred on the skin layer or muscle layer, other than the fat layer. To prevent this, a front end of the cannula 30, i.e. the tip portion, can be manufactured in a blunt shape or processed into a gently curved surface, so that the surgical site is not excessively damaged, with no penetration into the skin layer or muscle layer, etc., even when an inordinate amount of force is applied, up to a certain degree.

Also, during a liposuction surgery procedure, the surgeon can hold a joystick, etc., with the right hand to manipulate the robot, and touch the skin at the point where the tip portion of the cannula 30 is positioned with the left hand to feel whether there is skin, muscle, or fat in the current position, i.e. to check whether or not the robot is performing surgery at the correct position.

For this, an ultrasound transducer 37 can be coupled to an end portion of the cannula 30 according to this embodiment. With the ultrasound transducer 37 mounted on the front end of the cannula 30, it is possible to photograph the surgical site to provide visual information, and thus is possible to see the tissues that the end of the cannula 30 is touching.

Unlike a regular camera, an ultrasound transducer 37 can see the tissues of the surgical site even when there is no separate light source, and by using the characteristics of ultrasonic waves, the ultrasound transducer 37 can not only see the surface of a tissue but also see through the inside of the tissue, to differentiate the layers of fat tissues, skin tissues, muscle tissues, etc.

A surgical robot according to this embodiment can be driven by a surgeon manually manipulating a 3-dimensional joystick, etc., or can be controlled such that the suction unit 32 moves automatically to find a fat layer within a defined operating scope, or the operating scope can be pre-designated beforehand, so that the suction unit 32 only moves within this range.

Here, various control methods can be applied in designating the range of movement of the suction unit 32, i.e. the operating scope. For this embodiment, an ultrasound transducer 37 which photographs the surgical site to provide visual information can be installed, and the operating scope can be set based on the visual information obtained from the ultrasound transducer 37, so that the robot arm 20 can be driven to move the suction unit 32 within that range.

That is, the operating scope can be designated by marking on the screen with an input means such as a mouse or touchscreen, etc., while viewing the images taken by ultrasonic waves on a monitor screen.

The operating scope can also be designated by image-processing the visuals taken by ultrasonic waves to automatically differentiate fat tissues, skin tissues, and muscle tissues and detect the scope in which the fat layer is distributed.

Furthermore, the surgeon can input a particular range by driving the robot arm 20 beforehand, before inserting the cannula 30 into the body, and arrange the settings such that the robot arm 20 is driven only within the inputted range, so that the suction unit 32 is controlled to move only within the operating scope.

That is, before the operation, the cannula 30 can be mounted on the robot and moved manually, to input into the robot beforehand the range in which the cannula 30 can move, and during the operation, the robot arm 20 can be made to move only within the pre-inputted range. For example, a pen can be tied to the tip of the cannula 30, and a movement corresponding to drawing the range where liposuction is to be performed with the pen can be inputted, at which the robot may identify the movement, and the cannula 30 can be operated within the inputted range only.

The ultrasound transducer 37 used in this embodiment can provide not only visual information regarding the surgical site, but also non-visual information such as Doppler signals, etc. That is, not only is it possible to distinguish whether a particular portion is fat or muscle, etc., by analyzing visual information obtained by emitting ultrasonic waves, but it is also possible to sense the existence and positions of blood vessels by utilizing an ultrasound Doppler function.

The ultrasound Doppler function is for recognizing the flow speed of blood by using the frequency shift caused by the Doppler effect. It uses the property that, when blood flows towards the transducer, the frequency of the received echo is higher than the frequency of the emitted wave, and conversely, when blood flows away from the transducer, the frequency of the received echo is lower than the frequency of the emitted wave.

By using the ultrasound Doppler function in this manner, it is possible to sense beforehand any blood vessels present in the surgical site. Accordingly, by driving the robot arm 20 to minimize damage to the blood vessels, i.e. with the operating scope set such that the suction unit 32 avoids the blood vessels, it is possible to greatly reduce blood loss during a liposuction surgical procedure.

A user manipulation unit 40 can be connected to a surgical robot according to this embodiment, in which case the surgeon can control the robot arm 20 to move forward, backward, left, right, up, and down using a 3-dimensional joystick, etc., installed on the user manipulation unit 40. If the front end portion of the cannula 30 touches skin or muscle instead of fat when the surgeon is manipulating the robot, this can be sensed by the information obtained from the ultrasound transducer 37, and a suitable alarm signal, such as a warning sound, etc., may be supplied at the user manipulation unit 40 to alert the surgeon, so that the cannula 30 may not invade beyond a desired scope.

A surgical robot according to this embodiment can thus sensing skin, fat, and muscle automatically, so that the suction unit 32 can be controlled to move only in the fat layer. Occasionally, however, there may be instances of error in which, despite the surgeon's judgment that the suction unit 32 is positioned among fat tissues, the information obtained from the ultrasound transducer 37 causes the system to identify them as skin or muscle and prevent the robot arm 20 from advancing further.

To deal with this, a surgical robot according to this embodiment can also include an “override” function, which prioritizes signals received from user manipulation over signals received from the ultrasound transducer 37, i.e. enables the surgeon to forcibly operate the robot when necessary.

An ultrasound transducer 37 may be of a point-sensor type, which is able to see a 1-dimensional structure in front of the sensor, a line-sensor type, which is able to see a 2-dimensional structure in front of the sensor, or an area-sensor type, which is able to see a 3-dimensional structure in front of the sensor. The structure of the line-sensor type may include point sensors arranged in one direction, while the structure of the area-sensor type may include line sensors arranged in one direction.

An ultrasound transducer 37 according to this embodiment can be coupled to a front end of a cannula 30 mounted on a robot arm 20, and the robot arm 20 can move as the control unit 10 identifies information related to its position in real time. Thus, even when a point-sensor type or line-sensor type ultrasound transducer 37 is mounted on the front end of the cannula 30, moving the robot arm 20 up, down, left, and right allows a point-sensor type ultrasound transducer 37 to function as a line-sensor type and/or area-sensor type, or allows a line-sensor type ultrasound transducer 37 to function as an area-sensor type.

For example, by moving a point-sensor type ultrasound transducer up, down, left, and right, or moving a line-sensor type ultrasound transducer up, down, left, and right, it is possible to check the properties of the materials in front in a way that an area-sensor type ultrasound transducer does.

That is, even when the ultrasound transducer 37 according to this embodiment is not necessarily of an area-sensor type, the driving of the robot arm 20 allows it to function as an area sensor and obtain visual information of the materials in front.

Also, the operation of the suction unit 32 according to this embodiment, i.e. whether or not to perform suctioning and to what degree, can be controlled based on an analysis of the visual information obtained by ultrasound photography. Since the surgical robot according to this embodiment can automatically differentiate fat tissues, skin tissues, and muscle tissues, it can be controlled, for example, such that the suction unit 32 is operated only when the suction unit 32 is positioned within a fat layer.

The ultrasound transducer 37 according to this embodiment can also be used for “ultrasound-assisted liposuction.” That is, the ultrasound transducer 37 can emit high-energy ultrasonic waves, which generate heat. The fat at the surgical site may be liquefied by this heat and subsequently suctioned out.

As described above, an excessive movement of the cannula 30 during a liposuction procedure creates a risk of damage incurred on the skin layer or muscle layer other than the fat layer, and to prevent this, a reaction sensing unit (not shown), such as a strain gauge or a haptic sensor, etc., can be mounted on the tip of the cannula 30 or on another suitable position.

If a reaction sensing unit is mounted on the front end of the cannula 30, for example, the reaction force applied on the front end of the cannula 30 during a surgical procedure can be sensed. The control unit 10 can then receive the sensed signals, and if a force greater than a designated value is applied on the tip portion of the cannula 30, for example when the tip portion of the cannula 30 touches a skin tissue or a muscle tissue, can limit the drive range of the robot arm 20 so as not to further advance the cannula 30.

This uses the property that the reaction force applied on the cannula 30 differs according to the portion touched by the tip of the cannula 30, because the skin and muscle are considerably tough whereas fat tissues are relatively soft. As the reaction sensing unit detects the reaction force on the tip of the cannula 30, the surgical robot is able to automatically sense skin and muscle, whereby the liposuction operation can be performed with the robot arm 20 automatically moving the cannula 30.

Furthermore, a “force feedback” function can also be included, which applies a reaction force to a manipulation on the joystick, etc., installed on the user manipulation unit in accordance to a signal received from the reaction sensing unit, to recreate the sensation felt when the surgeon uses his/her own hands to operate. That is, a function can be installed by which the reaction sensing unit senses the touching of skin or muscle instead of fat by the front end portion of the cannula 30 and reflects the force onto the joystick, etc., and as the reaction force felt at the tip of the cannula 30 can be felt at the joystick also, the sensation felt when the surgeon operates with his/her own hands can be recreated.

A reaction sensing unit according to this embodiment can be mounted in a suitable position as necessary. If, for example, an existing cannula 30 is to be mounted as is onto the robot arm 20, the reaction sensing unit can be positioned with a haptic sensor installed at the point where the robot arm 20 holds the cannula 30, i.e. between the robot arm 20 and the cannula 30.

Alternatively, the reaction sensing unit can be positioned between the cannula and the cannula handle, i.e. at a middle portion of the cannula. It is also possible to implement an arrangement in which the cannula 30 itself has a built-in reaction sensing unit. For example, by having a reaction sensing unit, such as a strain gauge or a haptic sensor, etc., built in at the front end of the cannula 30 in an integrated form, the reaction sensing unit can be positioned such that the cannula 30 itself is able to measure the reaction force applied on its front end in accordance to the movement of the cannula 30.

A cannula 30 mounted on the robot arm 20 according to this embodiment can be an existing manual cannula used for liposuction that has been modified in structure to be mounted on the robot arm 20 or can also be manufactured as an exclusive cannula that has been designed in correspondence to the shape and structure of the robot arm 20.

The cannula 30 according to this embodiment can be structured to bend at a middle point, in which case the front end of the cannula 30, inserted through a hole perforated in the skin and bent, can be rotated freely without having to implement an RCM as in the case of a linear cannula 30, to suction fat from a larger scope.

The cannula 30 can be made to vibrate forward, backward, left, right, up, and down within a certain range, to maximize liposuction efficiency. To have the cannula 30 vibrate at a particular frequency, various methods can be applied, such as a method of adding a vibrating tool to the cannula 30, a method of implementing a vibrating function in the robot arm 20 on which the cannula 30 is mounted, and the like.

FIG. 11 is a flowchart illustrating a method of performing liposuction surgery according to an embodiment of the invention.

This embodiment relates to a method of performing surgery using a surgical robot for liposuction described above. First, the robot arm 20 mounted with the cannula 30 may be driven such that an end portion of the cannula 30, i.e. the suction unit 32, is inserted in the desired surgical site (W10).

Next, the fat at the surgical site may be suctioned through the suction unit 32 as a negative pressure is applied in the cannula 30 (W20). During this procedure, the robot arm 20 may be driven such that the suction unit 32 suctions fat while moving within a designated operating scope (W30), whereby the fat may be suctioned and removed at the desired site.

The range in which the suction unit 32 moves, i.e. the operating scope, can be automatically set by photographing the surgical site with an ultrasound transducer 37 to obtain visual information and analyzing the obtained image, as described above, whereby the surgical robot according to this embodiment can be made to detect a fat layer and perform liposuction surgery automatically.

The method of performing liposuction surgery described above can also be implemented in the form of a computer program that is readable and executable by a digital processing device, such as a microprocessor, etc., which may be built in inside the surgical robot itself or installed externally of the robot and connected with the robot.

While the present invention has been described with reference to particular embodiments, it is to be appreciated that various changes and modifications can be made by those skilled in the art without departing from the spirit and scope of the present invention as defined by the appended claims.

1.-89. (canceled)

90. A surgical robot for liposuction, the surgical robot comprising:
a control unit generating a control signal necessary for driving the surgical robot;
a robot arm driven by the control signal received from the control unit;
a cannula mounted on the robot arm, extending in one direction, and configured to be inserted up to a particular point through a hole perforated in a skin of a surgery patient;
a suction unit formed on an end portion of the cannula, the suction unit configured to be inserted into a surgical site to suction fat; and
a haptic sensor configured to measure a reaction force applied on a front end of the cannula,
wherein the robot arm is driven such that the particular point on the cannula is a RCM (remote center of motion), and
the control unit generates a control signal for driving the robot arm such that the suction unit moves within an operating scope set based on a signal received from the haptic sensor.

91. The surgical robot of claim 90, further comprising:
an image-taking camera configured to photograph substances suctioned by the suction unit and discharged out from a body; and
a computing unit configured to determine a degree of blood loss from data deduced by an image analysis of visual information obtained from the image-taking camera,
wherein the suction unit is configured to stop the operation in case where blood loss is determined to be greater than a certain degree by the computing unit.

92. The surgical robot of claim 90, further comprising:
an angle sensor configured to measure an angle by which the cannula is inserted,
wherein the control unit receives a signal from the angle sensor to generate a control signal for driving the robot arm such that the cannula remains in a particular angle range.

93. The surgical robot of claim 90, further comprising:
a chemical sensor configured to detect a chemical component of substances suctioned by the suction unit and discharged out from a body; and
a computing unit configured to determine a degree of blood loss from data obtained by the chemical sensor,
wherein the suction unit is configured to stop the operation in case where blood loss is determined to be greater than a certain degree by the computing unit.

94. The surgical robot of claim 90, wherein the control unit is connected to a user manipulation unit, and the control unit receives a signal from the haptic sensor to generate a control signal for causing the user manipulation unit to supply an alarm signal.

95. The surgical robot of claim 90, wherein the control unit is configured to perform an override function for prioritizing a signal received by user manipulation over a signal received from the haptic sensor.

96. The surgical robot of claim 90, wherein the control unit is connected to a user manipulation unit, and the control unit is configured to perform a force feedback function for applying a reaction force to a manipulation of the user manipulation unit, in correspondence to a signal received from the haptic sensor.

97. The surgical robot of claim 90, further comprising:
a storage unit configured to store data related to an amount of fat at a surgical site;
a volume sensor configured to measure an amount of fat suctioned by the suction unit and discharged out from a body; and
a calculating unit configured to deduce a degree to which surgery has progressed by comparing data stored in the storage unit with data measured by the volume sensor,
wherein the control unit generates a control signal for causing the robot arm and the suction unit to stop the operation in case where surgery is determined to be progressed to greater than a certain degree by the calculating unit.

98. The surgical robot of claim 90, wherein a front end of the cannula is processed as a gently curved surface so as not to damage a surgical site.

99. A surgical robot for liposuction, the surgical robot comprising:
a control unit generating a control signal necessary for driving the surgical robot;
a robot arm driven by the control signal received from the control unit;
a cannula mounted on the robot arm, extending in one direction, and configured to be inserted up to a particular point through a hole perforated in a skin of a surgery patient;
a suction unit formed on an end portion of the cannula, the suction unit configured to be inserted into a surgical site to suction fat; and a vision unit mounted on a front end of the cannula, and configured to photograph the surgical site to provide visual information,
wherein the robot arm is driven such that the particular point on the cannula is a RCM (remote center of motion),
the control unit generates a control signal for driving the robot arm such that the suction unit moves within an operating scope set from data deduced by an image analysis of the visual information, and
the suction unit is operated in case where the front end of the cannula is checked to be positioned among fat tissues from the visual information.

100. The surgical robot of claim 90, wherein a breaker unit is formed on an end portion of the cannula such that the breaker unit is rotated by a suction force applied in the cannula, the breaker unit is configured to break up fat suctioned through the suction unit.

101. The surgical robot of claim 99, wherein the control unit generates a control signal for driving the robot arm such that the suction unit moves within an operating scope set beforehand by a user.

102. The surgical robot of claim 90, wherein the cannula is structured to bend at a particular point on the cannula.

103. The surgical robot of claim 99, wherein a water jet unit configured to shoot water to a surgical site is coupled to an end portion of the cannula, and the water jet unit is operated in correspondence to the visual information.

104. The surgical robot of claim 99, further comprising:
a computing unit configured to determine a degree of blood loss from data deduced by an image analysis of the visual information,
wherein the suction unit is configured to stop the operation in case where blood loss is determined to be greater than a certain degree by the computing unit.

105. The surgical robot of claim 99, further comprising:
an image-taking camera configured to photograph substances suctioned by the suction unit and discharged out from a body; and
a computing unit configured to determine a degree of blood loss from data deduced by an image analysis of visual information obtained from the image-taking camera,
wherein the suction unit is configured to stop the operation in case where blood loss is determined to be greater than a certain degree by the computing unit.

106. The surgical robot of claim 99, further comprising:
a chemical sensor configured to detect a chemical component of substances suctioned by the suction unit and discharged out from a body; and
a computing unit configured to determine a degree of blood loss from data obtained by the chemical sensor,
wherein the suction unit is configured to stop the operation in case where blood loss is determined to be greater than a certain degree by the computing unit.

107. The surgical robot of claim 99, further comprising:
a storage unit configured to store data related to an amount of fat at a surgical site;
a volume sensor configured to measure an amount of fat suctioned by the suction unit and discharged out from a body; and
a calculating unit configured to deduce a degree to which surgery has progressed by comparing data stored in the storage unit with data measured by the volume sensor,
wherein the control unit generates a control signal for causing the robot arm and the suction unit to stop the operation in case where surgery is determined to be progressed to greater than a certain degree by the calculating unit.

108. The surgical robot of claim 99, wherein a breaker unit is formed on an end portion of the cannula such that the breaker unit is rotated by a suction force applied in the cannula, the breaker unit is configured to break up fat suctioned through the suction unit.

109. The surgical robot of claim 99, wherein the cannula is structured to bend at a particular point on the cannula.

110. The surgical robot of claim 99, wherein the cannula is configured to vibrate at a particular frequency so as to improve a suction efficiency of the suction unit.

111. The surgical robot of claim 90, wherein the cannula is configured to vibrate at a particular frequency so as to improve a suction efficiency of the suction unit.
